(19) United States
(12) Patent Application Publication
(10) Pub. No.: US 2023/0157389 A1
(43) Pub. Date: May 25, 2023

(54) POOL MASK AND MANUFACTURING METHOD FOR THE POOL MASK

(71) Applicant: Rockin' Pool Inc., Tokyo (JP)

(72) Inventor: Junya NISHIGAWA, Tokyo (JP)

(21) Appl. No.: 15/734,214

(22) PCT Filed: May 17, 2020
(86) PCT No.: PCT/JP2020/019563

§ 371 Date: Dec. 1, 2020

(30) Foreign Application Priority Data
Apr. 19, 2020 (JP) 2020-074457

(51) Int. Cl. A41D 13/11

(52) U.S. Cl. CPC A41D 13/1184

(57) ABSTRACT

Provided is a pool mask that can be used even in water in the field of swimming lessons, for example. The present invention provides a pool mask that, when worn by a user, forms a predetermined space between a main body part and at least a mouth of the user and is open downward so that the space communicates with an external space. To manufacture the pool mask, first, a right main body part and a left main body part that are formed of a polyvinyl chloride material are prepared. Next, an end of the right main body part and an end of the left main body part are sewed up together to form a central part. Finally, a strap part is attached to each of the other end of the right main body part and the other end of the left main body part.

### TECHNICAL FIELD

The present invention relates to a pool mask, and a manufacturing method for the pool mask.

### BACKGROUND ART

More people have come to wear a mask on the face in various fields including daily life, manufacturing sites in factories, construction sites, and health care sites.

Patent Literature 1 has disclosed a characteristic silicone rubber mask having a thickness of 0.5 mm, which is formed as a mask shape. From an outer surface of this mask, which is excellent in close contact property, cold resistance, and non-stickiness, new influenza virus can be removed easily and cooled oxygen can be sent into a nasal cavity.

Patent Literature 2 has disclosed a mask that is used by being worn on a face. A main body part of the mask that covers at least a part of the face includes a resin film having an air permeability in a thickness direction. The resin film is a non-porous film with a plurality of penetration holes extending in the thickness direction. The penetration hole has a diameter of 0.01 μm or more and 30 μm or less. The penetration hole in the resin film has a density of 10 holes/cm² or more and 1×10⁸ holes/cm² or less.

Patent Literature 3 has disclosed a facepiece mask that a user wears on the face. This mask has a plurality of pleats that are continuous so that the mask can expand and contract following any motion of the face, and moreover has a function of filtering the exhaled air.

Patent Literature 4 has disclosed as mask that is formed by: integrally molding a transparent synthetic resin material with suitable hardness and thickness into a mask shape in the range of covering eyes, a nose, and a mouth; providing a partition to the mask shape between a part under the nose and the upper lip; bonding an elastic and soft sheet to a part where an edge of the mask shape and the partition are in contact with the face, so as to cover a space that is formed at an uneven part of the face; and providing a hole for breathing at the nose and mouth parts and pasting a filter to those parts. This mask unifies the function of the conventional cloth mask and the function of goggles.

### CITATION LIST

### Patent Literatures

### Patent Literature 1: JP 2011-56210 A

### Patent Literature 2: JP 2017-2454 A

### Patent Literature 3: Japanese Patent No. 6145406

### Patent Literature 4: Utility Model Registration No. 3124550

### SUMMARY OF INVENTION

### Technical Problem

The aforementioned masks shield the nose and the mouth from the external environment, and when the air is taken in through the mask, intrusion of microparticles such as pollen or virus into a trachea or the like can be prevented or reduced.

Incidentally, in the field of swimming lessons, for example, a swimming instructor may spit saliva unintentionally in the lesson. In the swimming lesson, unlike any other sports, the instructor and those who take the lesson have their lines of sight at the similar height, and are close to each other. Thus, when the saliva touches the other person's mouth directly, for example, the disease may be transmitted. In particular, when a swimmer practices how to breathe, for example, he breathes out very powerfully and this may result in spitting of much saliva.

The mask according to any of Patent Literatures 1 to 4 described above is unusable in water, for example in a pool; therefore, the mask has been unable to prevent the dispersion of saliva in the pool.

In view of the above, an object of the present invention is to provide a pool mask that can be used even in water in the field of swimming lessons, for example.

### Solution to Problem

The present inventor has examined the use of a water-proof material for a general mask, which is the mask as described in Patent Literatures 1 to 4; however, covering the nose and mouth like the general mask results in a problem that the instructor's voice is not heard well. The present invention has been made on the basis of this knowledge.

That is to say, according to the present invention, a pool mask that, when worn by a user, forms a predetermined space between a main body part and at least a mouth of the user and is open downward so that the space communicates with an external space is obtained.

### Advantageous Effects of Invention

According to the present invention, the pool mask that can be used even in water in the fields including the swimming lessons can be provided. In addition, by the structure of the pool mask according to the present invention, those who take the swimming lesson can hear the instructor's voice clearly because the voice is not blocked by the mask.

In addition, since the mask is open downward, the splash of water does not stay in the main body part. Furthermore, if the swimmer puts his face in water for a moment and lifts up the face again, for example, the water in the main body part flows out of the mask from the opening below, and thus, the water does not remain in the main body part.

### BRIEF DESCRIPTION OF DRAWINGS

FIG. 1 is a diagram illustrating a pool mask according to an embodiment of the present invention.

FIG. 2 is a front view of a user who wears the mask illustrated in FIG. 1.

FIG. 3 is a diagram in which a state illustrated in FIG. 4 is viewed from the left side.

FIG. 4 is another diagram in which the state illustrated in FIG. 4 is viewed from the left side.

FIG. 5 is a diagram illustrating a positional relation between the mask in FIG. 1 and swimming goggles.

FIG. 6 is a diagram illustrating components of the pool mask in FIG. 1.

FIG. 7 is a diagram illustrating a state in which the components in FIG. 2 are sewed together to form the pool mask.

### DESCRIPTION OF EMBODIMENT

An embodiment of the present invention is described. The present invention includes the following aspects.

### [Aspect 1]

A pool mask that, when worn by a user, forms a predetermined space between a main body part and at least a mouth of the user and is open downward so that the space communicates with an external space.

### [Aspect 2]

The pool mask according to Aspect 1, in which the main body part is formed of a water-proof material.

### [Aspect 3]

The pool mask according to Aspect 1 or 2, in which the main body part is formed of a transparent material.

### [Aspect 4]

The pool mask according to any one of Aspects 1 to 3, in which

the main body part is formed of thermoplastic resin.

### [Aspect 5]

The pool mask according to Aspect 4, in which the main body part is formed of polyvinyl chloride.

### [Aspect 6]

The pool mask according to any one of Aspects 1 to 5, in which

the main body part has an upper end with a shape curved downward, and

the upper end is in contact with a lower part of goggles.

### [Aspect 7]

The pool mask according to any one of Aspects 1 to 6, including

a strap part that is attached to the main body part and includes chlorine-resistant rubber.

### [Aspect 8]

A manufacturing method for a pool mask that, when worn by a user, forms a predetermined space between a central part of a main body part and at least a mouth of the user and is open downward so that the space communicates with an external space, the manufacturing method including:

a step of preparing a right main body part and a left main body part that are formed of a polyvinyl chloride material;

a step of forming the central part by sewing together an end of the right main body part and an end of the left main body part; and

a step of attaching a strap part to the other end of the right main body part and the other end of the left main body part.

### Details of Embodiment

The embodiment of the present invention is hereinafter described with reference to drawings.

### SUMMARY

The pool mask according to the embodiment of the present invention is used mainly in the lessons in the pool, for example.

### <Structure of Pool Mask>

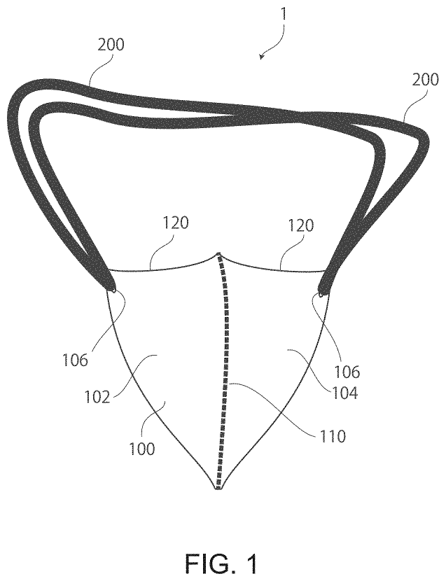

As illustrated in FIG. 1, the pool mask 1 according to the present embodiment includes a main body part 100 and a strap part 200. A right main body part 102 and a left main body part 104 are sewed up together along a seam part 110; thus, the main body part 100 is formed as one part.

Each of the right main body part 102 and the left main body part 104 includes a hole part 106 to which the strap part 200 is attached. The hole part 106 is formed by punching into a circular shape.

As illustrated in the drawing, an upper end of each of the right main body part 102 and the left main body part 104 has a shape curved downward, and serves as a contact part 120.

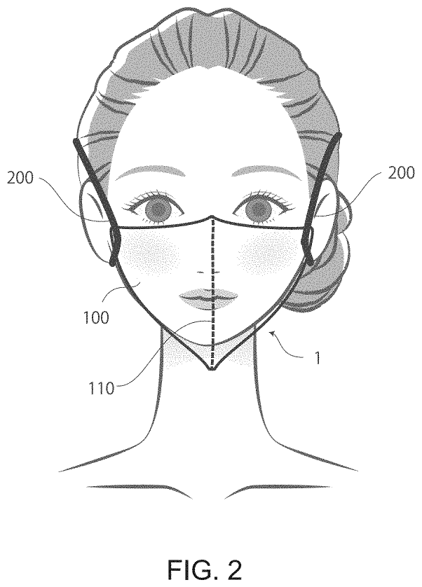

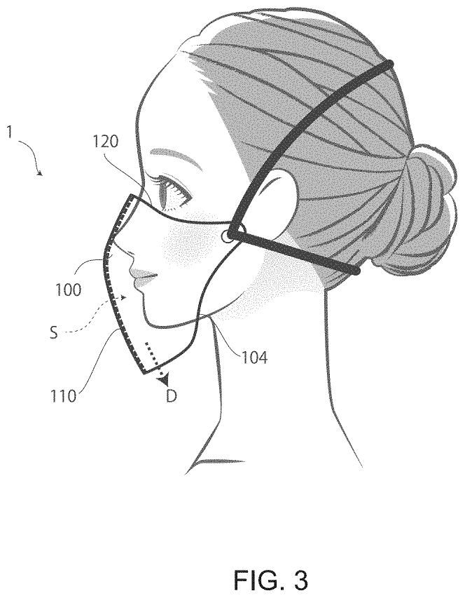

As illustrated in FIG. 2 and FIG. 3, when a user wears the pool mask 1 according to the present embodiment, a predetermined space S is formed between the main body part 100 and the user's mouth, and the pool mask 1 is open so that the space S and an external space communicate in a direction of an arrow D, that is, on the lower side.

The main body part 100 according to the present embodiment is used in water, for example in the pool, and therefore is made of a water-proof material. More specifically, the main body part 100 is formed of thermoplastic resin.

Examples of the thermoplastic resin include polyethylene, polypropylene, polyvinyl chloride, polystyrene, polyvinyl acetate, polyurethane, Teflon (registered trademark), ABS (acrylonitrile butadiene styrene) resin, and acrylic resin. The pool mask 1 needs to have hardness (strength) of such a degree that the space S as illustrated in FIG. 3 is formed; on the other hand, the pool mask 1 needs to be soft enough to fit the face line. Thus, the thickness and the density can be changed as appropriate. The pool mask 1 according to the present embodiment is formed of polyvinyl chloride in consideration of the hardness, the manufacturing cost, and the easiness of the manufacture.

As illustrated in FIG. 2, the main body part 100 is formed of a transparent material such as vinyl chloride. Thus, people can see how the instructor's mouth moves and the facial expression clearly. In particular, when the instructor who teaches children wears the pool mask 1, the children can feel comfortable. Furthermore, it is desirable that the pool mask 1 is transparent in order to know the color of the lips, for example. Note that the transparency can be changed as appropriate.

The strap part 200 that is attached to the main body part 100 is formed of chlorine-resistant rubber in order to avoid the influence of chlorine that is used in the pool. Examples of such a material include isoprene rubber, ethylene propylene rubber, chlorosulfonated polyethylene, natural rubber, butadiene rubber, acrylic rubber, chloroprene rubber, nitrile rubber, butyl rubber, and fluorine rubber. A string or the like made of normal resin other than the rubber material may be used if the size or the like is adjusted.

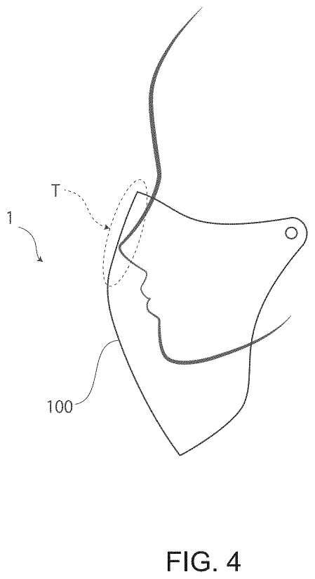

As illustrated in FIG. 4, the pool mask 1 is formed to have a shape of forming a region T where the pool mask 1 is in contact with a part of the user's face when the user wears the pool mask 1. The region T exists inside the main body part 100 above the center in the vertical direction. With this structure, the pool mask 1 is open downward and at the same time, an upper part thereof is in contact with a part of the face. Note that a part of the face and the main body part 100 may be in contact with each other partially.

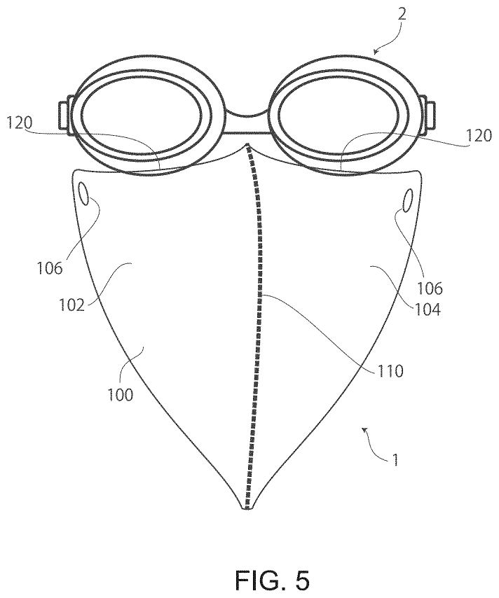

As illustrated in FIG. 5, the pool mask 1 according to the present embodiment may be structured so that when the user wears the pool mask 1, the contact part 120 is in contact with a lower part of swimming goggles 2. This structure can further prevent the pool mask 1 from being displaced upward.

By the aforementioned structure of the pool mask 1 according to the present embodiment, the pool mask 1 does not block the instructor's voice and those who take the lesson in the pool can hear the instructor's voice clearly. Since the pool mask 1 is open downward, the user who uses the pool mask 1 in the water can breathe normally because water does not remain in the main body part 100.

In particular, unlike the general mask, the main body part 100 of the pool mask 1 according to the present embodiment does not have a shape of covering below the chin as illustrated in FIG. 3. The pool mask 1 extends linearly downward while being kept away from the face line at a predetermined distance. In other words, the main body part 100 does not include a part that is in contact with the chin. By this structure, even if the main body part 100 is formed of thermoplastic resin or the like that does not expand or contract, speaking with the mouth wide open will not cause any pain because the material is not caught on the chin.

As thus described, the pool mask 1 according to the present embodiment does not completely block between the pool mask 1 and the face and shut out the external air but specifically, completely shuts out the splash of the saliva or the like that is spit forward from the user's mouth and nose.

### <Manufacturing Method for Pool Mask>

Subsequently, a manufacturing method for the pool mask 1 according to the present embodiment is described. That is to say, as described above, the pool mask that, when worn by the user (see FIG. 2 and FIG. 3), forms the predetermined space between the central part of the main body part 100 and at least the mouth of the user and is open downward so that the space communicates with the external space is manufactured as below.

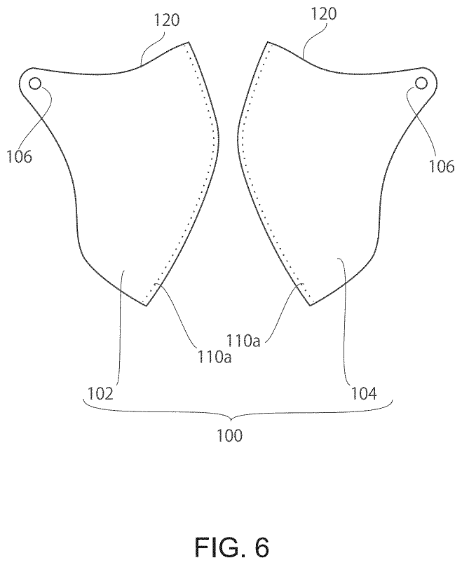

As illustrated in FIG. 6, the right main body part (part with which a right cheek is in contact when worn) 102 and the left main body part (part with which a right cheek is in contact when worn) 104 that are formed of the polyvinyl chloride material are prepared. The right main body part 102 and the left main body part 104 each have an approximately triangular shape (approximately convex shape) whose apex is the hole part 106. An end of the right main body part 102 and an end of the left main body part 104, which are opposite to ends thereof where the hole parts 106 are formed, each include a margin area 110a for sewing. Note that a mark (such as a dotted line) for sewing may be shown.

Subsequently, the margin area 110a of the right main body part 102 and the margin area 110a of the left main body part 104 are sewed up together to form the seam part as illustrated in FIG. 1. That is to say, after the right main body part 102 and the left main body part 104 are overlapped on each other, their margin areas 110a are sewed together with a sewing machine, for example. In this case, a presser foot of the sewing machine is preferably a Teflon (registered trademark) presser foot because the material advances smoothly. A sewing needle of the machine may be applied with silicon or the like.

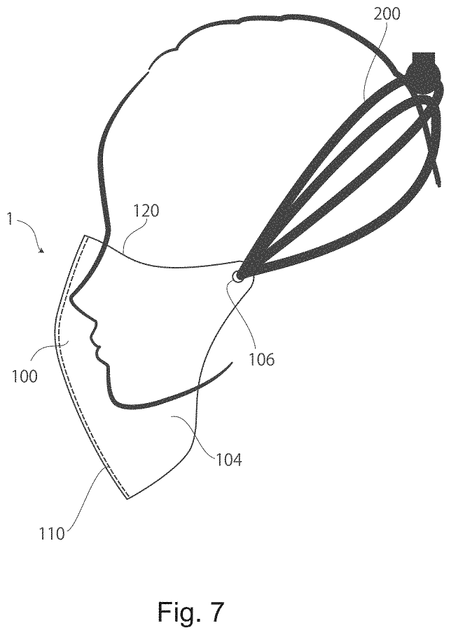

Finally, as illustrated in FIG. 7, the strap part 200 is put through the hole part 106 of the right main body part 102 and the hole part 106 of the left main body part 104.

Through the aforementioned procedure, the pool mask 1 according to the present embodiment is completed. The manufacturing method for the pool mask is not limited to this method and various methods are applicable. For example, the main body part 100 according to the present embodiment is formed by sewing the two materials of the right main body part 102 and the left main body part 104; however, the main body part 100 may be formed by integral molding with the use of a die or the like. That is to say, the manufacturing method is not limited to a particular method as long as the pool mask that, when worn by the user (see FIG. 2 and FIG. 3), forms the predetermined space between the central part of the main body part and at least the mouth of the user and is open downward so that the space communicates with the external space can be formed.

The aforementioned embodiment is just an illustrative example to make it easier to understand the present invention, and does not limit the scope of the present invention. The present invention can be changed or modified without departing from the concept, and the equivalents are included in the present invention.

### REFERENCE SIGNS LIST

1 Pool mask
2 Swimming goggles
100 Main body part
102 Right main body part
104 Left main body part
110 Seam part
120 Contact part
106 Hole part
200 Strap part

1. A pool mask that, when worn by a user, forms a predetermined space between a main body part and at least a mouth of the user and is open downward so that the space communicates with an external space.

2. The pool mask according to claim 1, wherein
the main body part is formed of a water-proof material.

3. The pool mask according to claim 1, wherein
the main body part is formed of a transparent material.

4. The pool mask according to claim 1, wherein
the main body part is formed of thermoplastic resin.

5. The pool mask according to claim 4, wherein
the main body part is formed of polyvinyl chloride.

6. The pool mask according to claim 1, wherein
the main body part has an upper end with a shape curved downward, and
the upper end is configured to be in contact with a lower part of swimming goggles.

7. The pool mask according to claim 1, comprising
a strap part that is attached to the main body part and includes chlorine-resistant rubber.

8. A manufacturing method for a pool mask that, when worn by a user, forms a predetermined space between a central part of a main body part and at least a mouth of the user and is open downward so that the space communicates with an external space, the manufacturing method comprising:
a step of preparing a right main body part and a left main body part that are formed of a polyvinyl chloride material;
a step of forming the central part by sewing together an end of the right main body part and an end of the left main body part; and
a step of attaching a strap part to the other end of the right main body part and the other end of the left main body part.
